(19) United States
(12) Patent Application Publication
(10) Pub. No.: US 2005/0123604 A1
(43) Pub. Date: Jun. 9, 2005

(54) Pharmaceutical compositions

(76) Inventors: Anees Abdulquadar Karnachi, Hillsborough, NJ (US); Maha Y. Khaled, Livingston, NJ (US); Jurij Holinej, Hackettstown, NJ (US); Yatindra Joshi, Princeton, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/506,821

(22) PCT Filed: Mar. 6, 2003
(86) PCT No.: PCT/EP03/02322

Related U.S. Application Data
(60) Provisional application No. 60/362,351, filed on Mar. 7, 2002.

(51) Int. Cl. A61K 31/195; A61K 9/20

(52) U.S. Cl. 424/464; 514/567

(57) ABSTRACT

A method of treating a cyclooxygenase-2 dependent disorder or condition comprising administering 5-methyl-2-(2′-chloro-6′-fluoroanilino)phenylacetic acid in an amount effective to treat such a disorder or condition for about 24 hours, comprising administering orally once a day to a human in need of such treatment one or more immediate release pharmaceutical compositions comprising 5-methyl-2-(2′-chloro-6′-fluoroanilino)phenylacetic acid, and compositions suitable for use in such methods.

This invention relates to compositions for the treatment of cyclooxygenase-2 mediated diseases and methods for stabilizing pharmaceutical compositions useful for the treatment of cyclooxygenase-2 mediated diseases.

In particular, this invention relates to compositions that comprise 5-methyl-2-(2′-chloro-6′-fluoroanilino)phenylacetic acid.

It has been surprisingly found that when the drug substance 5-methyl-2-(2′-chloro-6′-fluoroanilino)phenylacetic acid is formulated in solid form, e.g., in tablet form, the stability of the drug substance is increased by increasing the moisture content of the tablet, within a relatively narrow range, beyond which the stability decreases. That is, certain degradation products of 5-methyl-2-(2′-chloro-6′-fluoroanilino)phenylacetic acid are formed at a greater rate in dryer tablets, which is contrary to the typical behavior of drug substances. In general, moisture, in the form of water, is detrimental to drug stability, and packaging for pharmaceutical formulations frequently contains some form of desiccant material to minimize moisture levels.

This invention provides compositions for treating cyclooxygenase-2 dependent disorders or conditions comprising 5-methyl-2-(2′-chloro-6′-fluoroanilino)phenylacetic acid. The compositions comprise between about 200 and about 400 mg of 5-methyl-2-(2′-chloro-6′-fluoroanilino)phenylacetic acid and have a residual moisture level (“LOD”) between about 1.5% and about 5%. In certain embodiments, a composition comprising about 200 mg of 5-methyl-2-(2′-chloro-6′-fluoroanilino)phenylacetic acid will have an LOD between about 2% and 5%, or between about 2.1% and about 4.5%. In other embodiments, a composition comprising about 400 mg of 5-methyl-2-(2′-chloro-6′-fluoroanilino)phenylacetic acid will have an LOD between about 1.5% and about 4%, or between about 1.7% and about 3.5%. In certain embodiments, the compositions are tablets, and in other embodiments, film coated tablets.

In another aspect, the invention provides dried granulations useful for making pharmaceutical compositions. The dried granulations can comprise 5-methyl-2-(2′-chloro-6′-fluoroanilino)phenylacetic acid, microcrystalline cellulose, lactose monohydrate, and croscarmellose sodium, where the residual moisture level of the granulation is between about 2.5% and about 4.5%. The residual moisture level of the granulation can also be between about 3% and about 3.75%, e.g., about 3.5%. In another aspect, the invention provides dried granulations comprising 5-methyl-2-(2′-chloro-6′-fluoroanillno)phenylacetic acid, croscarmellose sodium, and povidone, where the residual moisture level of the granulation is between about 1.5% and about 4%, e.g., between about 1.7% and about 3.5%, e.g., between about 2% and about 3%, e.g., about 2.5%. The aforementioned granulations are useful in making tablets that contain, e.g., 100, 200, or 400 mg of 5-methyl-2-(2′-chloro-6′-fluoroanilino)phenylacetic acid, which tablets will have residual moisture levels corresponding to the level in the dried granulation used to make the tablet.

In another aspect, the invention provide methods for stabilizing 5-methyl-2-(2′-chloro-6′-fluoroanilino)phenylacetic acid in a pharmaceutical composition. The method comprises producing a solid pharmaceutical composition comprising 5-methyl-2-(2′-chloro-6′-fluoroanilino)phenylacetic acid, wherein the production yields a composition with a residual moisture level (“LOD”) between about 1.5% and about 5%. In certain embodiments, the method will yield a composition comprising about 200 mg of 5-methyl-2-(2′-chloro-6′-fluoroanilino)phenylacetic acid, which will have an LOD between about 2% and 5%, or between about 2.1% and about 4.5%. In other embodiments, the method will yield a composition comprising about 400 mg of 5-methyl-2-(2′-chloro-6′-fluoroanilino)phenylacetic acid that will have an LOD between about 1.5% and about 4%, or between about 1.7% and about 3.5%. In certain embodiments, the compositions produced are tablets, and in other embodiments, film coated tablets.

The pharmaceutical compositions useful in the practice of the invention are for oral administration and are “immediate release” dosage forms. That is, the pharmaceutical compositions useful in the practice of the invention have neither the pharmacokinetic nor physical characteristics of extended release pharmaceutical dosage forms. Thus, a pharmaceutical composition useful in the practice of the invention, if in solid form, will disintegrate or dissolve rapidly, preferably within one hour of administration, and administration of a pharmaceutical composition useful in the practice of the invention will result in a rapid rise in the blood plasma concentration of 5-methyl-2-(2′-chloro-6′-fluoroanilino)phenylacetic acid. Preferably, the blood plasma concentration of 5-methyl-2-(2′-chloro-6′-fluoroanilino)phenylacetic acid will reach a maximum within two to six hours after oral administration and will then fall rapidly due to the relatively short (3 to 6 hour) half life of 5-methyl-2-(2′-chloro-6′-fluoroanilino)phenylacetic acid.

Non-immediate release drug formulations, which are not within the scope of the present invention or used therein, include, inter alia, delayed release and sustained release formulations. Sustained release formulations may be further subdivided into prolonged release and controlled release formulations. Delayed release systems are those that use repetitive, intermittent dosing of a drug from one or more immediate-release units incorporated into a single dosage form. Examples of delayed release formulations include repeat-action tablets and capsules, and enteric-coated tablets where timed release is achieved by a barrier coating. Delayed release formulations do not produce or maintain uniform blood plasma concentrations of drug, but rather produce intermittent peaks and troughs in the blood plasma concentration of a drug, which are both desirably within the therapeutic range for the drug.

Sustained release drug formulations include drug formulations that achieve slow release of a drug over an extended period of time. If a sustained release formulation can maintain a constant drug concentration in the blood plasma, it is referred to herein as a “controlled release” formulation. If it does not maintain a constant concentration of drug in the blood plasma, but maintains the concentration of the drug in the therapeutic range for a longer period of time than would be achievable with an immediate release formulation, it is referred to herein as a “prolonged release” formulation. Thus, controlled release formulations maintain a relatively constant, peak blood plasma concentration of drug over an extended period of time, typically twelve to twenty four hours; the compositions of the present invention do not.

Typically, sustained release oral dosage formulations are based on a diffusion system, a dissolution system, and osmotic system, or an ion-exchange system.

In diffusion systems, the release rate of the drug is determined by its diffusion through a water-insoluble polymer. There are two types of diffusion devices: reservoir devices, in which a core of drug is surrounded by a polymeric membrane, and matrix devices, in which dissolved or dispersed drug is distributed uniformly throughout an inert polymeric matrix. Typical methods used to make reservoir-type devices include microencapsulation of drug particles and press-coating of whole tablets or particles. Generally, particles coated by microencapsulation form a system where the drug is contained in the coating film as well as in the core of the microcapsule. Some materials typically used as the water-insoluble coating, alone or in combination, are hardened gelatin, methyl or ethylcelluloses, polyhydroxymethacrylate, hydroxypropylcellulose, polyvinylacetate, and waxes.

Matrix devices are typically made by mixing drug with matrix material and then compressing the mixture into tablets. When using wax matrices, drug is generally dispersed in molten wax, which is then congealed, granulated, and compressed into cores. Matrix systems typically have a priming dose of drug coated onto the drug-matrix core. The major types of materials used in the preparation of matrix devices are insoluble plastics, hydrophilic polymers and fatty compounds. Plastic matrices include methyl acrylate-methyl methacrylate, polyvinyl chloride and polyethylene. Hydrophilic polymers include methylcellulose, hydroxypropylmethylcellulose and sodium carboxymethylcellulose. Fatty compounds include waxes such as carnauba wax and glyceryl tristearate.

Most dissolution type sustained release formulations are either encapsulated dissolution systems or matrix dissolution systems. Encapsulated dissolution formulations can be prepared either by coating particles or granules of drug with varying thicknesses of slowly soluble polymers or by microencapsulation. A common method of microencapsulation is coacervation, which involves the addition of a hydrophilic substance to a colloidal dispersion. The hydrophilic substance, which coats the suspended particles, can be selected from a wide variety of natural and synthetic polymers including shellacs, waxes, starches, cellulose acetate phthalate (or butyrate) or polyvinylpyrrolidone. Once the coating material dissolves, all of the drug inside the microcapsule is available immediately for dissolution and absorption, allowing drug release to be controlled by adjusting the thickness and dissolution rate of the coat. If three or four coating thicknesses are used in the microcapsules that comprise a formulation, drugs will be released at different, predetermined times to give a delayed-release, pulsatile effect. If a spectrum of thicknesses is employed, a more constant blood concentration of the drug can be achieved. Encapsulated particles can be compressed into tablets or placed into capsules.

Matrix dissolution sustained release formulations are prepared by preparing particles comprising drug and slowly soluble polymer particles. Such particles can be prepared by congealing drug with a polymer or wax and spray-congealing the particles or by cooling the drug-coating mixture and screening it. Alternatively, an aqueous dispersion method can be used, where a drug-polymer mixture is sprayed or placed in water and the resulting particles are collected. The drug-polymer particles are then compressed into tablets.

Formulations that rely on osmotic gradients have also been used to provide sustained release of drug. Typically, such formulations involve a membrane, permeable to water but not drug, that surrounds a core of drug. The membrane has a small delivery aperture. Water flows through the semipermeable membrane, dissolves drug, which is then pumped out of the formulation through the delivery aperture. Materials that can be used as a semipermeable membrane are polyvinyl alcohol, polyurethane, cellulose acetate, ethylcellulose, and polyvinyl chloride.

The immediate release formulations useful in the practice of the invention are intended for oral use and may be prepared according to any method known to the art for the manufacture of immediate release pharmaceutical compositions. Such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The excipients cannot be water soluble, water insoluble, or water permeable polymers or waxes where such water soluble, water insoluble, or water permeable polymers or waxes are present in an amount sufficient to impart a sustained release property to the formulation. In a most preferred embodiment, the immediate release pharmaceutical composition is a tablet.

5-methyl-2-(2′-chloro-6′-fluoroanilino)phenylacetic acid has surprisingly been found to undergo a variety of degradation processes when formulated as solid dosage forms, e.g., tablets. Tablets with about 200 mg of active agent preferably have an LOD of 3.5% with a desirable range between about 2.1% and about 4.5%. 65% drug-loaded tablets with about 400 mg of active agent preferably have an LOD of about 2.5%, with a desirable range between about 1.7% and about 3.5%. It has unexpectedly been found that if the LOD in the tablets are kept within the aforementioned parameters, the active agent, i.e., 5-methyl-2-(2′-chloro-6-fluoro-anilino)phenylacetic acid, is more chemically stable.

It has been surprisingly found that the ranges set forth above are the optimum LOD window between two different pathways by which 5-methyl-2-(2′-chloro-6′-fluoroanilino)phenylacetic acid degrades, i.e., an oxidative pathway and a cyclic pathway. The compositions and methods of the invention provide solid pharmaceutical compositions for oral administration comprising 5-methyl-2-(2′-chloro-6′-fluoroanilino)phenylacetic acid with minimal levels of total degradation products Oral dosage levels for 5-methyl-2-(2′-chloro-6′-fluoroanilino)phenylacetic acid are of the order of between about 200 and about 1200 mg per patient per day. In a preferred embodiment, the effective amount is between about 200 and about 800 mg. In a more preferred embodiment, the effective amount is between about 200 and about 600 mg. In an even more preferred embodiment, the effective amount is between about 200 and about 400 mg. In the most preferred embodiment, the effective amount is about 400 mg.

The amount of drug that may be combined with the carrier materials to produce a single dosage form will vary depending upon the size and weight of the recipient, the body composition of the recipient, and the particular mode of administration. For example, a formulation intended for oral administration by human recipients may contain between about 50 and about 1200 mg of agent compounded with an appropriate and convenient amount of carrier material which may vary from about 5 to about 95 percent of the total composition. Dosage unit forms may typically contain drug in amounts of 50, 100, 200, 300, 400, 600 or 800 mg. In one embodiment, the immediate release pharmaceutical composition comprises between about 50 and about 1200 mg of the 5-methyl-2-(2′-chloro-6′-fluoroanilino)phenylacetic acid. In a preferred embodiment, the immediate release pharmaceutical composition comprises between about 50 and about 600 mg of the 5-methyl-2-(2′-chloro-6′-fluoroanilino)phenylacetic acid. In a yet more preferred embodiment, the immediate release pharmaceutical composition comprises between about 50 and about 400 mg of the 5-methyl-2-(2′-chloro-6′-fluoroanilino)phenylacetic acid. In the most preferred embodiment, the immediate release pharmaceutical composition comprises about 400 mg of the 5-methyl-2-(2′-chloro-6′-fluoroanilino)phenylacetic acid. In a particular embodiment, the immediate release composition comprises a capsule or tablet. In another embodiment, the immediate release pharmaceutical formulation comprises a film-coated tablet.

Typically, the compositions of the invention comprise 5-methyl-2-(2′-chloro-6′-fluoroanilino)phenylacetic acid at a drug loading level of 50% to 90% by weight based on the weight of the composition.

In a particular aspect, this invention provides an immediate release tablet comprising about 400 mg of 5-methyl-2-(2′-chloro-6′-fluoroanilino)phenylacetic acid, wherein the tablet comprises between about 60% and about 70% of 5-methyl-2-(2′-chloro-6′-fluoroanilino)phenylacetic acid by weight. The immediate release tablet may comprise about 65% of 5-methyl-2-(2′-chloro-6′-fluoroanilino)phenylacetic acid by weight. In another aspect, the invention provides an immediate release tablet comprising about 200 mg of 5-methyl-2-(2′-chloro-6′-fluoroanilino)phenylacetic acid, wherein the tablet comprises about 50% of 5-methyl-2-(2′-chloro-6′-fluoroanilino)phenylacetic acid by weight.

It will be understood, however, that the specific dose level for any particular patient will depend upon a variety of factors including the age, body weight, general health, sex, diet, time of administration, rate of excretion, drug combination and the type and severity of the particular disease undergoing therapy. For many patients, a once daily dosage range of between about 200 and about 1200 mg. per day, or between about 200 and about 400 mg per day is indicated.

The invention provides in a further aspect a highly compressed tablet with a high drug loading. The tablet may be small in dimension e.g. 10 to 20 mm in diameter, preferably 15 to 20 mm, most preferably 17 to 18 mm; 5 to 10 mm in width, preferably 6.5 to 7.5 mm. The thickness of the tablet is from 4 to 8 mm, preferably 4.5 to 6.5 mm, most preferably 5.8 mm. Compression forces of between 10 to 20 kilo Newtons are used to prepare the compressed tablet. Benefits of this high drug loading include improved bioavailability, release characteristics and compliance.

Following is a description by way of example only of compositions of the invention.

### EXAMPLE 1

### Preparation of Formulations

TABLE 1

| | Amount per 200 mg |
|---|---|
| Ingredient | tablet batch (kg) |
| Core | |
| Granulation | |
| 5-methyl-2-(2′-chloro-6′- | 50** |
| fluoroanilino)phenylacetic acid drug | |
| substance | |
| Microcrystalline cellulose, NF (PH | 12.85 |
| 101) | |
| Lactose monohydrate, NF | 11.65 |
| Croscarmellose sodium, NF | 1 |
| Povidone, USP | 4 |
| Titanium dioxide, USP | 2 |
| Water, purified***, USP | 20.375 |
| Extra-granular Phase | |
| Microcrystalline cellulose, NF (PH | 13 |
| 102) | |
| Croscarmellose sodium, NF | 3 |
| Titanium dioxide, USP | 2 |
| Magnesium stearate, NF | 0.5 |
| Coating | |
| Opadry white | 2.801**** |
| Opadry yellow | 2.0**** |
| Opadry red | 0.4**** |
| Opadry black | 0.0504**** |
| Water, purified***, USP | 29.758**** |

**The weight of drug substance is taken with reference to the dried substance (100 percent) on the basis of the assay value (factorization). The difference in weight is adjusted by the amount of microcrystalline cellulose used.
***Removed during processing.
****Includes a 50% excess for loss during the coating process.

Table 1, above, sets out the formula for a batch of approximately 250,000 immediate release film-coated tablets of 5-methyl-2-(2′-chloro-6′-fluoroanilino)phenylacetic acid. To make the tablets, titanium dioxide is dispersed in water, followed by the addition of povidone and mixing for 20 minutes to make a povidone/titanium dioxide suspension. The drug substance, lactose, microcrystalline cellulose, and croscarmellose are mixed in a high shear mixer (e.g., a Collette Gral) for 5 minutes to form a drug mixture. The drug mixture is granulated in the high shear mixer with the povidone/titanium dioxide suspension. The suspension is pumped at a rate of 3 kg/min into the drug mixture. The resulting mixture is mixed an additional 90 seconds after all the suspension is added. The wet granulation is dried in a fluid bed dryer, using an inlet air temperature of 50° C., to form a dried granulation. The residual water target in the dried granulation is 3.5% (with an acceptable range of 2.1-4.5%). The dried granulation is passed through a screen using a mill (oscillator) and a 30 mesh screen. The previous steps are repeated to make a second dried granulation.

The extra-granular phase titanium dioxide is passed through a 60 mesh hand screen. The dry granulations are mixed with the extra-granular phase microcrystalline cellulose, croscarmellose sodium and titanium dioxide in a twin shell mixer for 300 revolutions to form a penultimate mixture. Magnesium stearate is passed through a 60 mesh hand screen and is mixed with the penultimate mixture in a twin shell mixer for 50 revolutions to form a tableting mixture. The tableting mixture is pressed into tablets using a tablet press and oval punches.

The coating powders (Opadry) are mixed with purified water to make a 15% w/w coating suspension. The tablets are film coated with the coating suspension in a coating pan using 60° C. to 75° C. inlet air temperature. Table 2 sets out the contents of a 200 mg 5-methyl-2-(2′-chloro-6′-fluoroanilino)phenylacetic acid film-coated tablet.

TABLE 2

| | Theoretical | |
|---|---|---|
| Ingredient | amount [mg] | Function |
| Core | | |
| 5-methyl-2-(2′-chloro-6′- | 200 | Active |
| fluoroanilino)phenylacetic | | substance |
| acid drug substance | | |
| Microcrystalline cellulose | 51.4 | Filler |
| (PH 101) | | |
| Lactose | 46.6 | Filler |
| Povidone | 16 | Binder |
| Titanium dioxide | 8 | Color |
| Croscarmellose sodium | 4 | Disintegrant |
| Water, purified* | Q.S. | Granulating |
| | | liquid |
| Extragranular phase | | |
| Microcrystalline cellulose | 52 | Filler |
| (PH 102) | | |
| Croscarmellose sodium | 12 | Disintegrant |
| Titanium dioxide | 8 | Color |
| Magnesium stearate | 2 | Lubricant |
| Core weight | 400 | |
| Coating | | |
| Opadry white (00F18296) | 7.4676 | Color |
| Opadry yellow (00F12951) | 5.3312 | Color |
| Opadry red (00F15613) | 1.0668 | Color |
| Opadry black (00F17713) | 0.1344 | Color |
| Water, purified* | Q.S. | Coating solvent |
| Total weight | 414 | |

*removed during processing

A tablet with 400 mg drug substance can be formulated as follows:

TABLE 3
400 mg formulation composition

| % w/w | Ingredient | Mg/dose | Kg/batch |
|---|---|---|---|
| | Granulation | | |
| 65.04 | Drug substance | 400.00 | 20.00 |
| 2.15 | Croscarmellose sodium, NF (Ac-Di-Sol) | 13.22 | 0.661 |
| 6.60 | Povidone K30, USP | 40.59 | 2.029 |
| 18.12 | Purified water, USP* | Qs | Qs |
| | Blending | | |
| 23.56 | Microcrystalline Cellulose, NF | 144.90 | 6.066 |
| | (Avicel PH 102) | | |
| 2.15 | Croscarmellose sodium, NF (Ac-Di-Sol) | 13.22 | 0.553 |
| 0.50 | Magnesium Stearate, NF (vegetable | 3.07 | 0.128 |
| | source) | | |
| | Film Coating | | |
| 84.46 | Opadry, Global White 00F18296 | 15.2028 | 0.296637 |
| 14.03 | Opadry, Global Red 00F15613 | 2.5254 | 0.049275 |
| 1.51 | Opadry, Global Black 00F17713 | 0.2718 | 0.005303 |
| | Purified Water, USP* | Qs | 1.990218 |
| | Film Coated Tablet Weight | 633.00 | |

*Does not appear in final product. Percentage of water added used for granulation based on the dry weight of drug substance and croscarmellose sodium.

The tablets are formulated by first mixing the polyvinylpyrrolidone binder with water, followed by addition of the drug substance and croscarmellose sodium to the povidone solution. This mixture is granulated in a Gral mixer. The resulting granulation is dried in a fluid bed dryer to yield a dried granulation with an LOD of about 2.5%, with an acceptable range of 1.7% to 3.5%, and is screened over an oscillating 18 mesh screen. Microcrystalline cellulose (Avicel PH-102, NF) is mixed with croscarmellose sodium and the resulting mixture is screened over an 18 mesh screen. The screened mixture is blended with the screened, dried granulation of polyvinylpyrrolidone, drug substance, and croscarmellose sodium. The resulting mixture is then blended with magnesium stearate that has been screened through an 18 mesh screen. The resulting blend is then compressed on a tablet press.

All patents, patent applications, and other publications referred to herein are hereby expressly incorporated by reference in their entirety. In case of a conflict between the present specification and material incorporated by reference, the present specification is controlling.

1. A solid pharmaceutical composition for treating a cyclooxygenase-2 dependent disorder or condition comprising between about 200 and about 400 mg of 5-methyl-2-(2′-chloro-6′-fluoroanilino)phenylacetic acid, wherein the residual moisture levels of said composition is between about 1.5% and about 5%.

2. The solid pharmaceutical composition of claim 1 comprising about 200 mg of 5-methyl-2-(2′-chloro-6′-fluoroanilino)phenylacetic acid, wherein the residual moisture level of said composition is between about 2% and about 5%.

3. The solid pharmaceutical composition of claim 2 wherein the residual moisture level of said composition is between about 2.1% and about 4.5%.

4. The solid pharmaceutical composition of claim 3, wherein the residual moisture level of said composition is about 3.5%.

5. The solid pharmaceutical composition of claim 1 comprising about 400 mg of 5-methyl-2-(2′-chloro-6′-fluoroanilino)phenylacetic acid, wherein the residual moisture level of said composition is between about 1.5% and about 4%.

6. The solid pharmaceutical composition of claim 5 comprising about 400 mg of 5-methyl-2-(2′-chloro-6′-fluoroanilino)phenylacetic acid, wherein the residual moisture level of said composition is between about 1.7% and about 3.5%.

7. The solid pharmaceutical composition of claim 6, wherein the residual moisture level of said composition is about 2.5%.

8. The solid pharmaceutical composition of claim 3, wherein said composition is a tablet.

9. The solid pharmaceutical composition of claim 4, wherein said composition is a tablet.

10. The solid pharmaceutical composition of claim 6, wherein said composition is a tablet.

11. The solid pharmaceutical composition of claim 7, wherein said composition is a tablet.

12. A method for stabilizing 5-methyl-2-(2′-chloro-6′-fluoroanilino)phenylacetic acid in a solid pharmaceutical composition, comprising producing a solid pharmaceutical composition comprising 5-methyl-2-(2′-chloro-6′-fluoroanilino)phenylacetic acid wherein said composition has a residual moisture level between about 1.5% and about 5%.

13. The method of claim 12, wherein said solid pharmaceutical composition comprises about 200 mg of 5-methyl-2-(2′-chloro-6′-fluoroanilino)phenylacetic acid, and wherein the residual moisture level of said composition is between about 2% and about 5%.

14. The method of claim 13 wherein said solid pharmaceutical composition comprises about 200 mg of 5-methyl-2-(2′-chloro-6′-fluoroanilino)phenylacetic acid, and wherein the residual moisture level of said composition is between about 2.1% and about 4.5%.

15. The method of claim 14 wherein said solid pharmaceutical composition comprises about 200 mg of 5-methyl-2-(2′-chloro-6′-fluoroanilino)phenylacetic acid, and wherein the residual moisture level of said composition is between about 3% and about 4%.

16. The method of claim 15 wherein said solid pharmaceutical composition comprises about 200 mg of 5-methyl-2-(2′-chloro-6′-fluoroanilino)phenylacetic acid, and wherein the residual moisture level of said composition is about 3.5%.

17. The method of claim 12, wherein said solid pharmaceutical composition comprises about 400 mg of 5-methyl-2-(2′-chloro-6′-fluoroanilino)phenylacetic acid, and wherein the residual moisture level of said composition is between about 1.5% and about 4%.

18. The method of claim 17 wherein said solid pharmaceutical composition comprises about 400 mg of 5-methyl-2-(2′-chloro-6′-fluoroanilino)phenylacetic acid, and wherein the residual moisture level of said composition is between about 1.7% and about 3.5%.

19. The method of claim 18 wherein said solid pharmaceutical composition comprises about 400 mg of 5-methyl-2-(2′-chloro-6′-fluoroanilino)phenylacetic acid, and wherein the residual moisture level of said composition is between about 2% and about 3%.

20. The method of claim 19 wherein said solid pharmaceutical composition comprises about 400 mg of 5-methyl-2-(2′-chloro-6′-fluoroanilino)phenylacetic acid, and wherein the residual moisture level of said composition is about 2.5%.

21. A dried granulation comprising 5-methyl-2-(2′-chloro-6′- fluoroanilino)phenylacetic acid, microcrystalline cellulose, lactose monohydrate, croscarmellose sodium, wherein the residual moisture level of said granulation is between about 2.5% and about 4.5%.

22. The dried granulation of claim 21, wherein the residual moisture level of said granulation is between about 3% and about 3.75%.

23. The dried granulation of claim 22, wherein the residual moisture level of said granulation is about 3.5%.

24. A dried granulation comprising 5-methyl-2-(2′-chloro-6′-fluoroanilino)phenylacetic acid, croscarmellose sodium, povidone, wherein the residual moisture level of said granulation is between about 1.5% and about 4%.

25. The dried granulation of claim 24, wherein the residual moisture level of said granulation is between about 1.7% and about 3.5%.

26. The dried granulation of claim 25, wherein the residual moisture level of said granulation is between about 2% and about 3%.

27. The dried granulation of claim 26, wherein the residual moisture level of said granulation is about 2.5%.

28. A solid pharmaceutical composition comprising the dried granulation of claim 21.

29. A solid pharmaceutical composition comprising the dried granulation of claim 22.

30. A solid pharmaceutical composition comprising the dried granulation of claim 25.

31. A solid pharmaceutical composition comprising the dried granulation of claim 26.
